(19) United States
(12) Patent Application Publication
(10) Pub. No.: US 2019/0311542 A1
(43) Pub. Date: Oct. 10, 2019

(54) SMART OPERATING ROOM EQUIPPED WITH SMART SURGICAL DEVICES

(71) Applicants: Robert E. Douglas, Winter Park, FL (US); Kathleen M. Douglas, Winter Park, FL (US); David Byron Douglas, Winter Park, FL (US)

(72) Inventors: Robert E. Douglas, Winter Park, FL (US); Kathleen M. Douglas, Winter Park, FL (US); David Byron Douglas, Winter Park, FL (US)

(21) Appl. No.: 15/949,202

(22) Filed: Apr. 10, 2018

(51) Int. Cl. G06T 19/00; G06T 7/33; G06F 3/01; A61B 6/00; A61B 34/10; G01S 5/10

(52) U.S. Cl. CPC G06T 19/006; G06T 7/33; G06F 3/012; A61B 6/466; A61B 34/10; A61B 2090/365; G06T 2207/30204; G06T 2207/30096; G06T 2207/20221; G06T 2207/10081; G06T 2207/10088; G01S 5/10

(57) ABSTRACT

Interactions between a surgical device and a patient are planned and monitored using data from a smart operating room to calculate the spatial location of the surgical device, both within the patient and within a patient 3D data set that includes a virtual representation of the patient. Virtual patient images are generated to enhance the surgeon's visualization of the progress of the operation. The images can be displayed, on the command of the surgeon, on a head display unit. For example, the images may be superimposed on the surgeon's real-world view with coordinated alignment such that virtualized aspects of the operation such as a planned incision can be viewed in their real-world location and orientation from any distance and angle.

### TECHNICAL FIELD

Aspects of this disclosure are generally related to surgery, and more specifically the operating room setup and surgical devices.

### BACKGROUND

The traditional operating room consists of personnel including the surgeon, anesthesiologist, nurses, and technicians, and equipment including the operating room table, bright lights, surgical instrumentation, supporting system equipment. Surgical instruments are directly manually controlled by the surgeon.

More recently, robotic surgical systems have been developed where the surgeon indirectly manually controls surgical instruments, such as cutting, cauterizing, suction, knot tying, etc., through robotic arms. Advantages may include smaller incisions, decreased blood loss, and shorter hospital stays. These techniques are gaining more acceptance in the operating room because of the advantages.

Stereotactic surgery is a technique for locating targets of surgical interest within the body relative to an external frame of reference using a 3D coordinate system. As an example, stereotactic neurosurgery has traditionally used a mechanical frame attached to the patient's skull or scalp, such that the head is in a fixed position within the coordinate system of the stereotactic device. In more recent techniques, patients undergo imaging exams (e.g., computed tomography (CT) scans) with a stereotactic frame or stereotactic markers placed onto reference points on either the skin or skull in place during the imaging examination. This establishes the patient's anatomy and the stereotactic reference points all within the same 3D coordinate system. Through stereotactic neurosurgery, precise localization can be performed, such as placement of a deep brain stimulator (DBS) leads placed through a small hole in the skull into a specific structure deep within the brain to treat Parkinson's Disease. In other surgeries, when the surgeon positions a probe inside the skull, the tip of the probe will register to a particular spot on the patient's image, which is helpful for surgical guidance.

Although the technological developments described above offer some advantages, there are several shortcomings associated with the modern day operating room and modern stereotactic surgical techniques. First, the 3D coordinate system only pertains to surgical devices that can be affixed to the frame. Free-standing objects separated from the stereotactic unit cannot be registered into the 3D coordinate system. Second, the 3D coordinate system only works for tissues that are immobile and non-deformable within the body (e.g., brain within rigid skull). Stereotactic system would not work for a mobile, deformable anatomic structure such as a breast; thus, precision procedures must be performed with constant image-guidance (e.g., MRI, CT, ultrasound) to account for the changing position and deformation of the breast tissue. Third, the volumetric 3D coordinate system of the patient's imaging study (e.g., MRI of a brain mass) is not manipulated in real time during the surgery in accordance with the expected ongoing surgical changes. As a result, there is a mismatch between the patient's surgical anatomy and the pre-operative imaging, which gets worse and worse as the surgical anatomy changes, such as removal of a glioma.

### SUMMARY

All examples, aspects and features mentioned in this document can be combined in any technically possible way.

In accordance with an aspect an apparatus comprises a geo-registration and operating system within a hospital or clinic surgical setting to precisely locate points within the setting in an operating room coordinate system. Some implementations comprise, but are not limited to: precisely placed transmitters at 6 or more locations within the operating room. Some implementations further comprise transmitters in radio frequency (RF) or in the electro-magnetic (EM) spectrum. Some implementations further comprise transmitters could emit a unique signal within the frequency band or transmit in differing frequencies together with a schedule for transmission and a receiver for the transmitted signals coupled with a differential timing system to reflect the precise location within the operating room coordinate system of the receiver of any point within the operating room. Such a system would allow numerous objects to be registered into the same 3D coordinate system including both free standing objects and objects mounted to stereotactic devices, such as the following: operating room table; stereotactic markers on the patient's skin; stereotactic markers planted within the patient's tissues; key anatomical patient landmarks; surgeon's augmented reality headset; surgeon's cutting and dissecting device; surgical instruments; and, many types of surgical devices.

Some implementations of the geo-registration system a patient coordinate system is established wherein small (e.g., pin head size) pieces of material which will provide a distinct signature in a medical image (e.g., MRI, CT) are affixed to the patient. These pieces would be placed at locations on the body, which surround the area of a surgical procedure (i.e., at least 6 locations). Under this implementation, medical images would be obtained and 3D data generated and placed into a patient coordinate system and which geo-locates the pieces of material within the patient coordinate system.

Some implementations of the geo-registration system further comprise an external pointing system containing an inertial motion sensor which can be moved to and the tip of the pointer touch each of the pieces of material and the tip of which is thereby located within the patient coordinate system and a computational system within the pointing system which tracks the location of the tip of the pointer in relation to the patient 3D data and within the patient coordinate system.

Some implementations of the geo-registration system further comprise registration of the Head Display Unit (HDU) which would have an inertial motion sensor. Then the surgeon would while wearing the HDU register the location and pointing angle of the HDU by centering the head over the intended cut area and converging the focus point of the eyes on three different of the small (e.g., pin head size) pieces of material which will provide a distinct signature in a medical image affixed to the patient. The readings from the inertial motion sensor would be transmitted to the processor and through intersection/resection the location and pointing angle would be computed.

Some implementations in connection with an operating room coordinate system further comprise registration of 3D patient data and associated patient coordinate system are geo-located with geo-registration system of the operating room (i.e., patient moved from medical imaging system to the operating room and the geo-location of each voxel of the patient 3D medical image is then converted to a geo-location within the operating room.) The receiver in the surgical setting could be moved to each of the pieces of material described in the patient coordinate system and the patient coordinate system then registered within the operating room geo-registration system.

Some implementations further comprise a pre-planning surgical process wherein the surgeon views the 3D volume containing the region for the operation and the pre-planning surgical process consists of, but not limited to: designating the volume of tissue on which the operation will be performed (e.g., tumor to be extracted); delineating the cutting surface within the region to access the designated volume of tissue for the operation; projecting the cutting surface to the external surface of the body from where the cutting will begin; taking note of and designating any areas for potential concern which are in close proximity to the cutting surface; obtaining metrics as of key elements of the surgery (e.g., depth of cut; proximity to arteries, veins, nerves); and recording the above for recall and display during the course of the operation.

Some implementations further comprise, in connection with the geo-registration and operating system a surgical device (e.g., but not limited to a scalpel with associated electronics) system with points along edge located with geo-registration system consisting of: if the conditions operating room coordinate system apply, then the surgical device would have a receiver for the transmitted signals coupled with a differential timing system to reflect the precise location of a precise point of the surgical device within the operating room; or if the patient coordinate system conditions apply, then the surgical device system would have the capability to compute the precise location of a precise point of the surgical device system within the patient coordinate system.

Some implementations further comprise the surgical device system would contain an inertial motion sensor which would measure roll, pitch and yaw of the surgical device and from that compute the precise location of the precise point of the surgical device, and the surgical device geometry (i.e., distance of the point of the surgical device from the precise point and also location of surgical device edge relative to precise point) compute the location of the various portions of the surgical device (e.g., point and edge) at any point in time within either operating room coordinate system or the patient coordinate system.

Some implementations further comprise a near real time communication system which transmits data from the surgical device system (i.e., key point on surgical device plus roll, pitch, and yaw) to the processor unit.

Some implementations further comprise a processing system which simultaneously computes surgical device location to include all cutting edges and its location within the patient 3D data.

Some implementations further comprise a near real time geo-locating system which tracks and records movements of the surgical device as it moves thru the patient and simultaneously through the patient 3D data.

Some implementations further comprise a head display system on/off (e.g., heads-up display which can be seen through when off and displays selected visual material when on) at direction of surgeon.

Some implementations further comprise a control system (e.g., audio from the surgeon or processor interface unit by surgeon's assistant) through which the surgeon can control what is to be displayed.

Some implementations further comprise at the start of the operation, the surgeon could select to display: patient with data considered relevant to surgeon (e.g., surgery type and objective; patient condition; the pre-planned cut line (length and planned depth) projected onto the patient; notes collected during the planning on any areas for potential concern which are in close proximity to the cutting surface).

Some implementations further comprise a process to compare the tracked movements of the surgical device with the planned cutting surface consisting of: a display of actual cutting surface vs. planned cutting surface on the surgeon's head display unit; metrics to inform the degree of variation of actual vs. planned; computation of needed angular approach (yaw, pitch and roll of cutting edge of surgical device) to arrive at the volume of tissue on which the operation will be performed; feedback to surgeon showing degree and direction of angular movements required to correct the variation of actual vs. planned cutting surface.

Some implementations further comprise deformable (e.g., breast, liver, brain, etc.) tissue (i.e., repositioning and/or resizing/reorienting of original voxels) within the patient 3D data to reflect pull back of tissue to access the designated volume of tissue for the operation as function of width of the pull back and depth of surgical cut and the type(s) of tissue involved.

Some implementations further comprise non-deformable (e.g., bone) tissue (i.e., repositioning/reorienting of voxels without resizing) within the patient 3D data to reflect movement of tissues to access the designated volume of tissue for the operation as a function of the surgical maneuver.

Some implementations further comprise of the placement of a surgical apparatus into the patient with the corresponding 3D representation of the surgical device being placed into the 3D patient imaging dataset.

Some implementations further comprise a process for color coding the deformable tissue to: reflect proximity of the cutting edge of the surgical device to volume of tissue on which the operation will be performed; or reflect distance to any areas of potential concern which are in close proximity to the cutting surface.

Some implementations further comprise an application of a variable degree of transparency of deformable tissue to enable viewing organs in proximity to the surgical cut.

Some implementations further comprise a display of metrics during the course of the operation to: show distances from cut to designated volume of tissue for the operation; show distances to areas of potential concern which are in close proximity to the cutting surface; and key organs and to surgical target for operation.

Some implementations further comprise the capability to display the intended cutting surface and also the actual cutting surface. In the event that there is a deviation between the planned and actual cutting surfaces wherein a corrective course is deemed appropriate, then the corrective angles and/or movement direction for the surgical device are calculated and displayed on the HDU.

Some implementations further comprise the capability to incorporate and display advice from an Artificial Intelligence (AI) program on the HDU. The AI program could be called by the surgeon. For example, if an artery were severed, the surgeon could ask the AI program for corrective actions.

Some implementations further comprise isolation of the tissue intended for the operation and present in 3D to the surgeon during planning for and conduct of an operation which would include, but not limited to the following anatomical sites: brain; head and neck structures; chest; abdomen; pelvis; and, extremities.

Some implementations further comprise for a tumor type of operation, encapsulate the tissue for the operation and some additional margin of tissue to ensure all tissue of concern has been retrieved.

Some implementations further comprise performing segmentation on the encapsulated tissue to distinguish between tissue of concern and benign tissue (per U.S. patent application Ser. No. 15/904,092). Some implementations further comprise removing benign tissue leaving only tissue of concern. Some implementations further comprise determining points, within the 3D data set containing the tissue of concern, those points closest to the left eye viewing point and those closest to the right eye viewing point (note this results in a convex surface point toward the surgeon). This could be replicated from multiple angles, resulting in a 3D volume which represents the outer surface of the tissue of concern. Some implementations further comprise, at the direction of the surgeon, performing a smoothing operation on the above volume to remove artifacts in the volume. Some implementations further comprise displaying the volume on the surgeon's head mounted display (HMD) together with metrics to show the size of this tissue.

Some implementations further comprise for a heart type of operation using the 3D data set, separate the heart into two pieces such that the internal structure within the heart can be viewed in 3D with the surgeon's HMD. Some implementations further comprise, using metrics, calculation of the volumes of the left and right atriums and left and right ventricles. Some implementations further comprise encapsulation of each of the heart valves for 3D display on surgeon's HMD; and, as required, use segmentation to remove extraneous tissue.

Some implementations further comprise a process to generate a real time medical imaging dataset. The starting point for such a dataset is the patient's pre-operative images. As the surgery progresses, the medical imaging dataset will be updated. As an example, as tissues are removed, they can be analyzed (e.g., size, shape, weight) and the surgical cavity can be analyzed (e.g., measure cavity by laser range finder to generate 3D map of the surgical cavity). A corresponding volume of the 3D medical imaging dataset will be removed, such that the medical imaging data is updated. Alternatively, hardware can be added into the operating bed. A corresponding digital 3D representation of the surgical device will be inserted into the medical images with voxels manipulated accordingly to account for the new volume. The resultant volume will represent a working copy of the estimated 3D medical imaging dataset and will be available to the surgeon in real time.

Some implementations further comprise a process for stacking imaging slices to generate a movable volume, which can be then filtered, segmented and rendered.

Some implementations further comprise a process for generating a 4D cursor, with the dimensions comprising length, width, height and time.

Some implementations further comprise a process for generating a multi-dimensional (5D or higher) cursor, which would include length, width, time, and tissue property(ies).

Some implementations further comprise a recording of surgical device and its cutting-edge locations in conjunction with the patient 3D data during the course of the operation.

In accordance with an aspect an apparatus comprises: a plurality of spatial locators adapted to be used in an operating room; a medical image registration device configured to use information from the spatial locators to register at least one medical image with respect to a human body in the operating room that will undergo a surgical procedure; and a display that presents the registered medical image.

In accordance with an aspect a method comprises: receiving data from a plurality of spatial locators adapted to be used in an operating room; using the data from the spatial locators to register at least one medical image with respect to a human body in the operating room that will undergo a surgical procedure; and presenting the registered medical image on a display

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIG. 1 illustrates a smart operating room in accordance with some aspects of the invention.

FIG. 2 depicts an example setup for a smart operating room which has an internal coordinate system.

FIG. 3 illustrates placement of registration markers on a patient.

FIG. 4 illustrates determination of the location and orientation of the surgical device (SD) within the smart operating room coordinate system.

FIG. 5 illustrates a patient coordinate system.

FIG. 6 illustrates axial, sagittal, and coronal views of the patient 3D data with the location and orientation of the SD within the 3D data set.

FIG. 7 illustrates the starting point, length, and depth of an incision as seen through the surgeon's augmented reality headset.

FIGS. 8A and 8B illustrate a surgical incision and tissue displacement along the cutting surface to reach the target.

FIG. 9 illustrates the exposed deformable tissue from a top view as seen through the surgeon's augmented reality headset.

FIG. 10 illustrates a variable degree of transparency that can be selected so that the surgeon can peer through the deformable tissue and see other portions of the anatomy in the general region of the cut through the surgeon's augmented reality headset.

FIG. 11 illustrates metrics available during an operation, such as depth of cut, as seen through the surgeon's augmented reality headset.

FIG. 12 illustrates the planned cutting surface vs. the actual cutting surface as seen through the surgeon's augmented reality headset.

FIG. 13A through 13E illustrate encapsulation and review of tissue of concern/tissue which is the objective of the operation.

FIG. 14 illustrates a process for generating a real-time imaging dataset to better approximate the current surgical anatomy with reference to FIGS. 15A through 15D.

FIGS. 16 through 18 illustrate stacking of slices to generate a mobile volume.

FIG. 19 illustrates a 4D cursor.

FIG. 20 illustrates a 5+ multidimensional cursor.

### DETAILED DESCRIPTION

Some aspects, features and implementations described herein may include machines such as computers, electronic components, optical components, and processes such as computer-implemented steps. It will be apparent to those of ordinary skill in the art that the computer-implemented steps may be stored as computer-executable instructions on a non-transitory computer-readable medium. Furthermore, it will be understood by those of ordinary skill in the art that the computer-executable instructions may be executed on a variety of tangible processor devices. For ease of exposition, not every step, device or component that may be part of a computer or data storage system is described herein. Those of ordinary skill in the art will recognize such steps, devices and components in view of the teachings of the present disclosure and the knowledge generally available to those of ordinary skill in the art. The corresponding machines and processes are therefore enabled and within the scope of the disclosure.

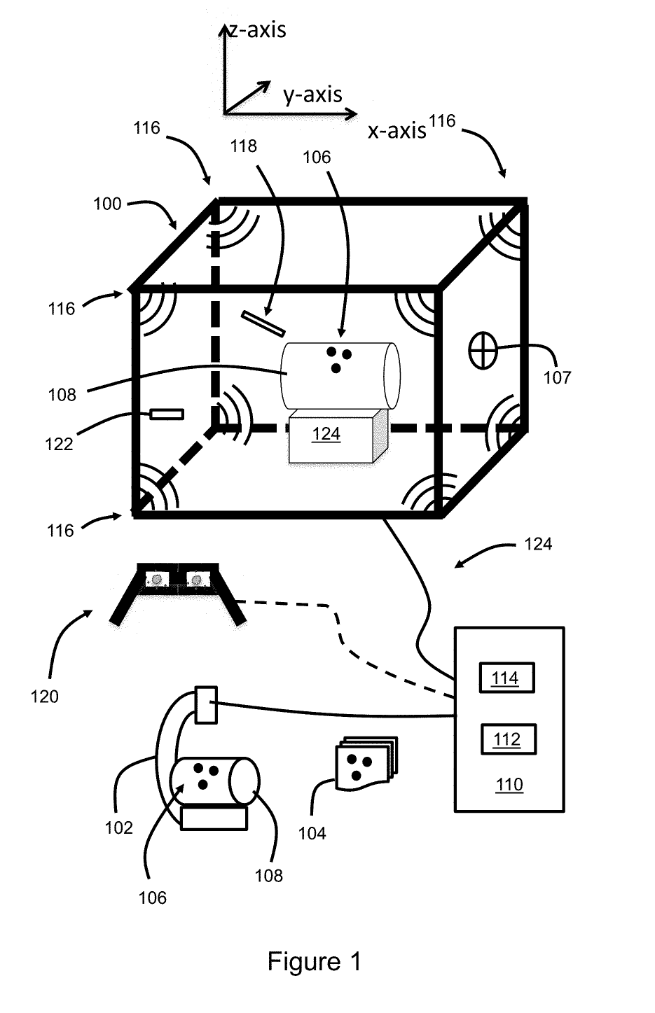

FIG. 1 illustrates a smart operating room 100 in accordance with some aspects of the invention. Aspects of an operation including interactions between components such as a surgical device (SD) 118 and a patient 108 are planned, monitored, and facilitated using a medical image registration computer 110. The computer uses data from spatial locators in the smart operating room to calculate the spatial location and orientation of the surgical device 118, both within the patient 108 and within a patient 3D data set 114 that includes a virtual representation of the patient. The 3D data set is registered with respect to the surgical device and the body of the patient. Virtual images of completed and planned surgical procedures are generated to enhance the surgeon's visualization of the progress of the operation. The virtual images can be displayed, on the command of the surgeon, on a HDU (head display unit) 120, e.g. an augmented reality headset. For example, the virtual images may be superimposed on the surgeon's real-world view with coordinated alignment such that virtual aspects of the operation can be viewed in their real-world locations and orientations from any distance and angle.

A radiological imaging instrument 102 is used to obtain medical images 104 prior to the operation. Reference point markers 106, which are readily recognizable in the medical images 104, are placed on the patient 108 prior to taking the images. The reference points would typically be proximate to, or surround, the locus of the operation, and may be placed on surfaces with little anticipated movement. The medical images 104, which may include multiple 2D slices, are provided to the computer 110. The computer may include processors, memory, non-volatile storage, and a control elements program 112 for processing the medical images 104 to help generate the patent 3D data set and perform other functions that will be described below.

The surgeon performs a pre-surgery planning process which may include a thorough review of the: patient data; objectives of the prospective operation; planning the operation cut(s); and delineation the cut parameters (e.g., cut location; depth); designation of areas of concern; device(s) to be placed; and a digital shape (e.g., sphere) around the tissue to be operated on. These plans are then entered into the patient 3D data set 114 and saved as a pre-surgical planning file on the computer 110.

The patient 108 is transported from the radiology room to the smart operating room 100 in preparation for surgery. The gurney 124 with the patient may be aligned with the long side of a rectangular room. Both the patient 108 and surgical device are spatially registered with respect to the patient 3D data set 114. A wide variety of other things may be registered with respect to the patient 3D data set, including both free standing objects and objects mounted to stereotactic devices, including but not limited to the following: operating room table; stereotactic markers on the patient's skin; stereotactic markers planted within the patient's tissues; key anatomical patient landmarks; the HUD 120; and many types of surgical devices. Spatial location within the smart operating room may be based on one or both of inertial motion sensors and the time-of-flight of signals transmitted between transmitter/receiver pairs. In one example the difference between transmission and receipt of signals 116 emitted by transmitters precisely located within the operating room and receivers located in or on the patient and/or surgical device 118, and/or HUD 120, and/or other things being registered are used to calculate distances, each of which defines a sphere, and multiple spheres are used to calculate precise spatial locations within the operating room. In another example a pointer 122 with an inertial motion sensor is used to spatially locate patient and/or surgical device 118, and/or HUD 120, and/or other things being registered using reference points with respect to at least one fixed registration point 107 in the smart operating room. For example, the pointer 122 may be placed in contact with the registration point 107 and then placed in contact with one of the reference point markers 106 on the patient, and then the inertial motion data may be used to calculate the location of the reference point marker with respect to the registration point. Similarly, the inertial motion sensor equipped surgical device and HUD could be initialized by being placed in contact with the registration point, or the surgeon would, while wearing the HDU, register the location and pointing angle of the HDU by centering the head over the intended cut area and converging the focus point of the eyes on three different of the small (e.g., pin head size) pieces of material which will provide a distinct signature in a medical image affixed to the patient. The readings from the inertial motion sensor would be transmitted to the processor and through intersection/resection the location and pointing angle would be computed. Utilizing both inertial motion sensing data and receiver/transmitter pair distance data may provide even more precise and reliable spatial location. The raw spatial location data may be converted to an X, Y, Z location in the operating room coordinate system. Spatially locating each of the reference points, e.g. at differing orientations/pointing positions and directions of point, establishes a patient coordinate system.

As will be explained in greater detail below, at the start of the operation the surgeon can prompt display of the planned cut in an image superimposed on the patient 108, together with notes prepared during the pre-planning process. Furthermore, the planned cut can be displayed in the surgeon's augmented reality headset 120, providing stereoscopic imaging since the headsets provide unique images to each eye. In one implementation the images are displayed in accordance with USPTO #8,38,4771, which is incorporated by reference. During the operation, progress can be displayed both in metrics with respect to distance of the cut from the tissues to be operated on and distances to areas of concern. Also, if the surface of the actual cut varies from the intended cut surface, alerts can be given to the surgeon and needed redirection movements of the surgical device displayed.

Finally, at the end of the operation, selected data can be automatically stored and/or inserted into a surgery report on the computer 110.

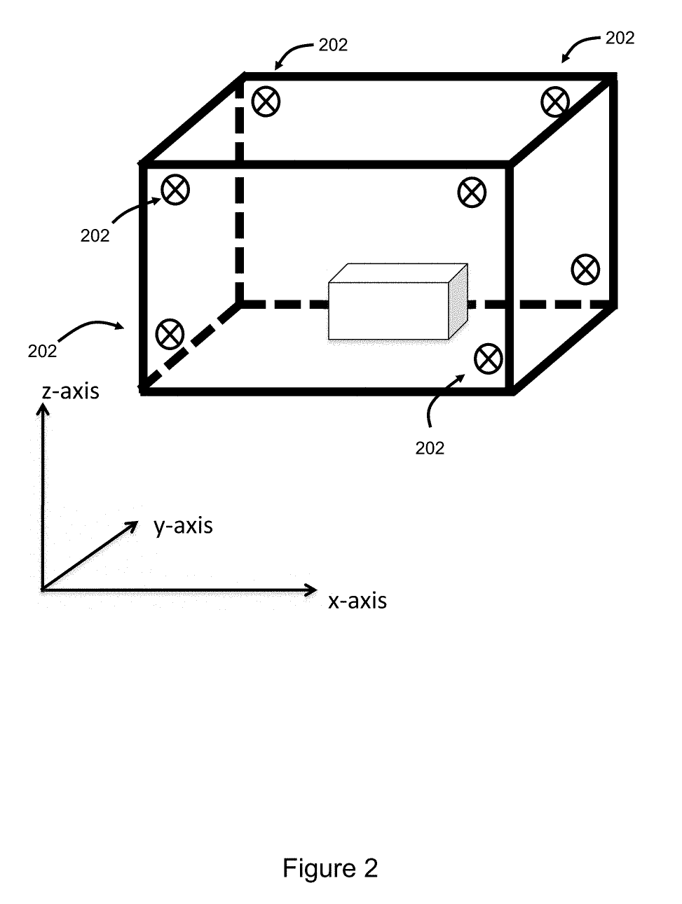

FIG. 2 depicts an implementation of the smart operating room with an internal coordinate system. In the illustrated example, six or more transmitters (or receivers) 202 are placed at specific locations within the room where they will not interfere with the operation. Distances between all possible pairs of transmitters are measured with appropriate precision, e.g. and without limitation to the nearest millimeter. A coordinate system may be established that is unique to the operating room. For purposes of illustration, the X axis is in the long direction of a rectangular cuboid room; the Y axis is the shorter horizontal dimension, and the Z axis is the vertical (height) dimension. TDM (time division multiplexing) FDM (frequency division multiplexing) and other techniques may be used for the transmitted signals. For example, each transmitter (or receiver) 202 may emit (or receive) a signal according to a specified schedule. The signals 116 (FIG. 1) could be all of the same frequency in the EM spectrum but with different pulse characteristics, or of differing frequencies. One or more receiver (or transmitter) elements, e.g. reference point markers 106 (FIG. 1), receive (or transmit) the signals. Duration of the transmission between transmitter/receiver pairs is used to calculate distances between transmitters and receivers. For example, and without limitation, the emitted signals may include a transmit time stamp that can be compared with a received time stamp to calculate signal flight time based on the time delta between the timestamps. The time difference can be used to calculate a corresponding unit of length distance from the transmitter based on the speed of the signal. Each calculated length distance may define a sphere, and intersections of spheres from multiple transmitters may be used to pinpoint the location of each receiver element. Thus, the patient and a variety of other things including the surgical device can be spatially located within the operating room, and registered with respect to the 3D patient data set.

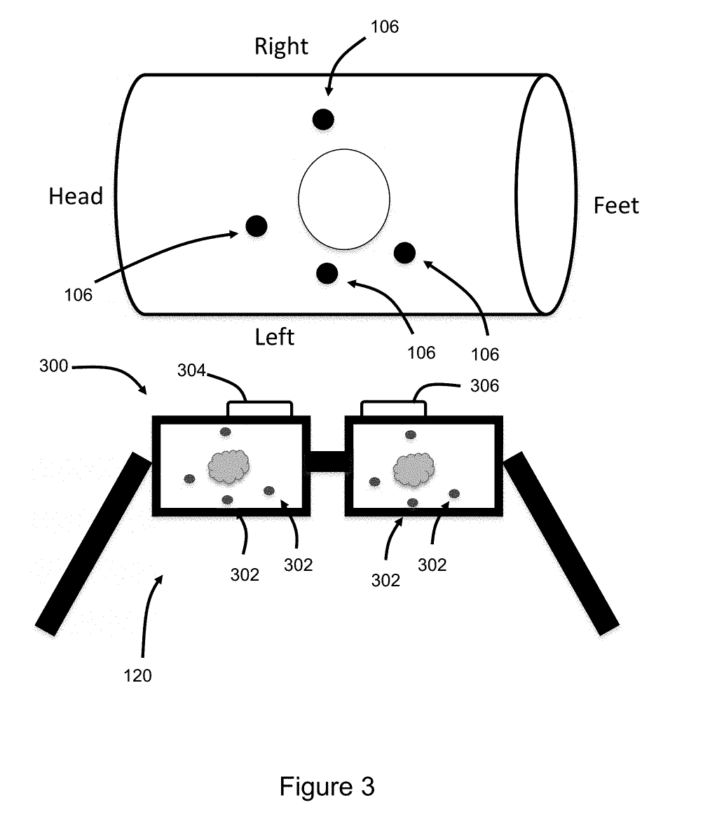

FIG. 3 depicts emplacement of the reference point markers 106. Note that the number of reference point markers depicted in the example is not limiting; a minimum of six of reference point markers that provide spatial location must be used, but a larger number might be used. In order to attain the optimum registration of the reference point markers with the patient's 3D imaging dataset, the reference point markers should be positioned prior to the imaging examination. When the surgeon wears the augmented reality headset 120, the surgeon can see the actual reference point markers 106 in the real-world view and an image 300 that includes virtual reference point markers 302. The augmented reality headset 120 may be a free-standing object with a transceiver 304 for communication and inertial motion sensor system 306. It would display the images in a depth-3-dimensional fashion, such that true 3D imaging is performed with depth perception.

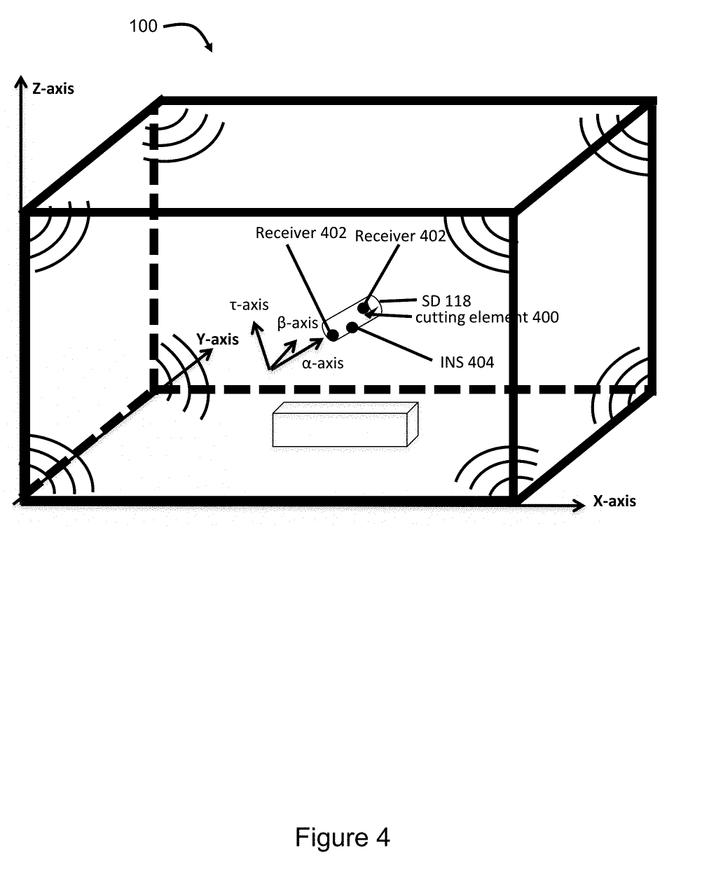

FIG. 4 depicts spatial location of the surgical device 118 within the coordinate system of the smart operating room 100. The system precisely calculates the spatial location (including orientation) of a cutting element 400 of the surgical device 118, and calculates and plots the trajectory of the cutting element at any point in time (actual trajectory before the current time and anticipated trajectory after the current time) both within the patient and within the patient 3D data set. Two or more receiver (or transmitter) elements 402 are positioned at non-cutting portions of the surgical device 118 to facilitate determination of spatial location of the surgical device. The location of each receiver element (X, Y, and Z coordinates) within the operating room is determined, and angles α, β, and τ are computed relative to the X, Y, Z axes, respectively. Based on the calculated spatial location of the surgical device, and the known dimensions of the surgical device and cutting element, cutting edge coordinates are thereby known. Roll of the surgical device may be calculated using data from the inertial motion sensor 404 and the known geometry of the surgical device. The surgical device 118 continuously transmits data from its inertial motion sensor and (receivers if the operating room coordinate system is being used) via the communication system. The computer continuously tracks the surgical device and generates various display options. When a particular display is selected by the surgeon, the computer sends the display to the HDU via the communications system. Thus, an incision can be monitored and forecast in three dimensions with respect to the patient.

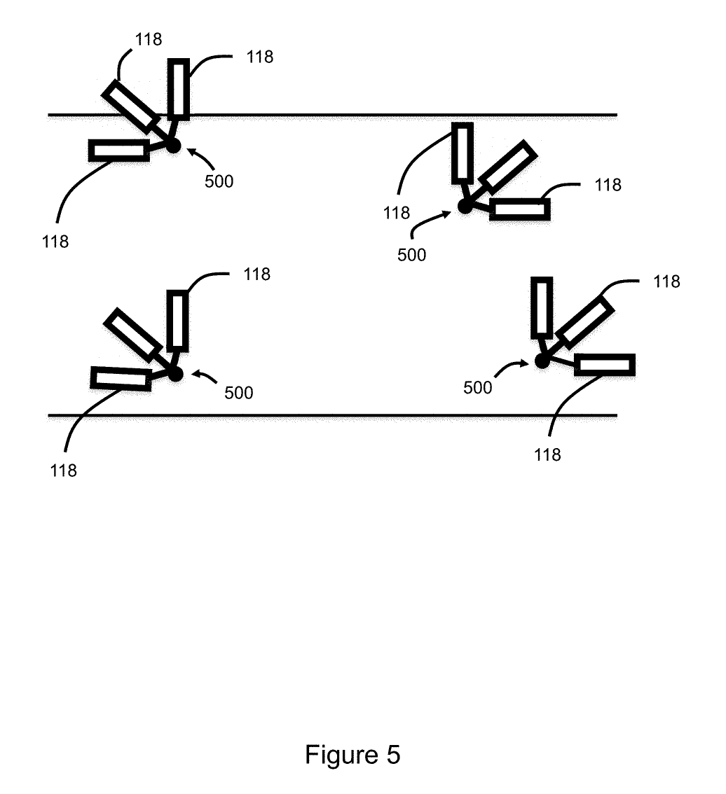

FIG. 5 depicts a patient coordinate system. To register the surgical device 118 with respect to registration points 500, e.g. reference point markers 106 (FIG. 3), the surgical device is positioned in contact with each of the registration points from three approximately perpendicular angles representing the X, Y, and Z axis, respectively. By convention, the X axis could be parallel to the length of the patient; Y axis the horizontal width of the patient; and Z axis the depth or height above the operating gurney. Note that the region within the patient for the operation is within the overall volume encased by the registration points. Only four registration points are shown on this figure whereas a minimum of six points is required for the registration process in practice.

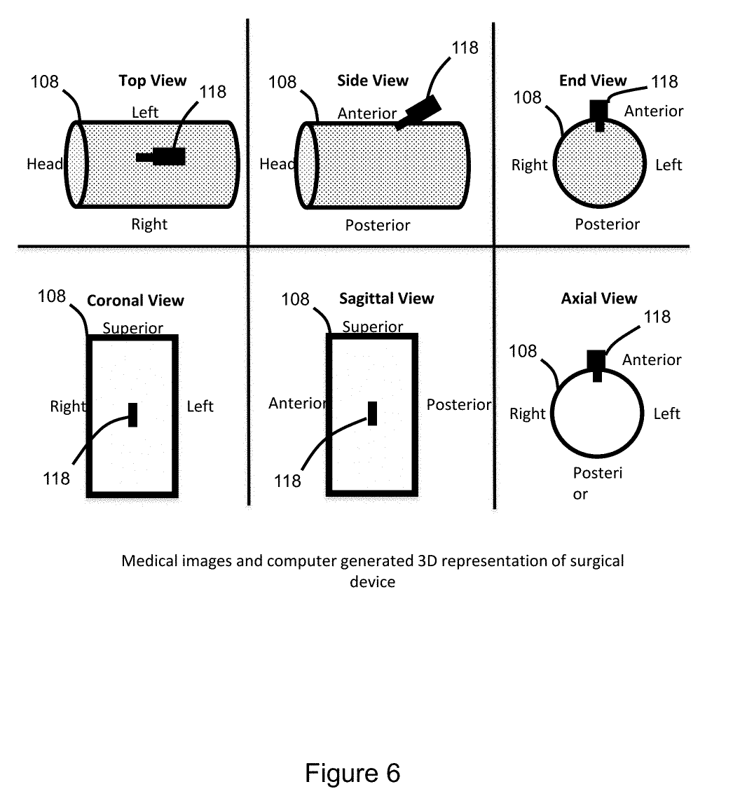

FIG. 6 illustrates spatial location of the surgical device 118 with reference to three views of the patient 108 (i.e., top, side, end) and three views of the 3D medical imaging data (i.e., axial, sagittal and coronal) with the location of the surgical within the 3D data set. Note that a 3D representation of a surgical device 118 is generated and superimposed onto the patient imaging dataset. These views could be displayed individually or collectively at any time at the direction of the surgeon. An option would be to show only a line showing the current location of the cutting edge of the surgical. Options to also display areas of concern and the shape containing tissue to be operated on could be displayed.

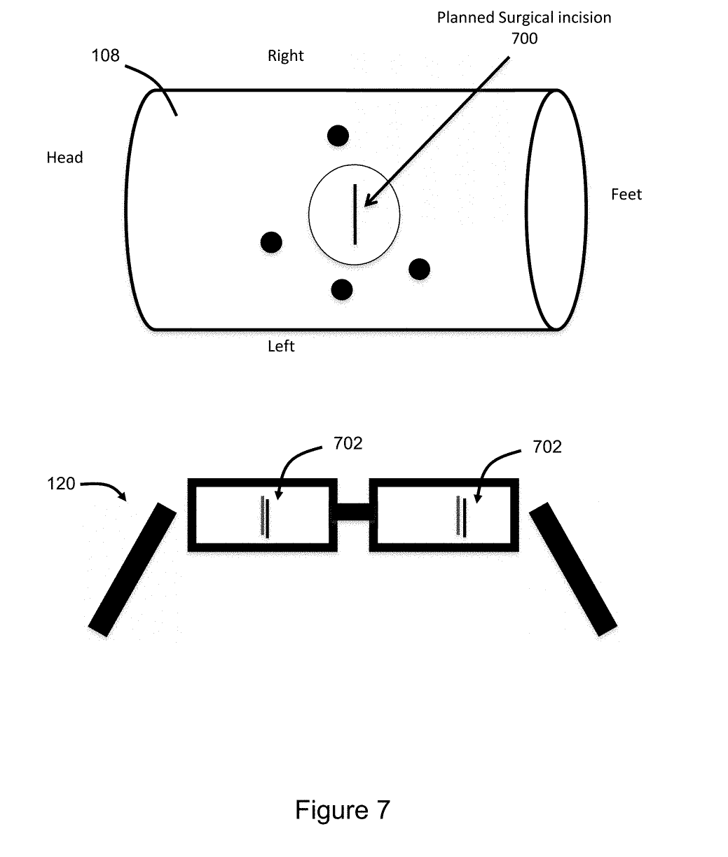

FIG. 7 depicts presentation of a planned surgical incision 700 on the HDU 120. A virtual incision 702 may indicate a starting point, length, and depth of a surgical incision that is a product of the pre-operative planning. The virtual incision may be presented on the surgeon's HDU 120 as a line (or other shape) superimposed on the patient 108 (i.e., from the stored pre-operative planning data within the patient 3D data set). Notes reflecting pre-operative planning may also be presented, e.g., proximity to regions of concern in red, whereas green indicates a planned cutting plane or surface of the virtual incision.

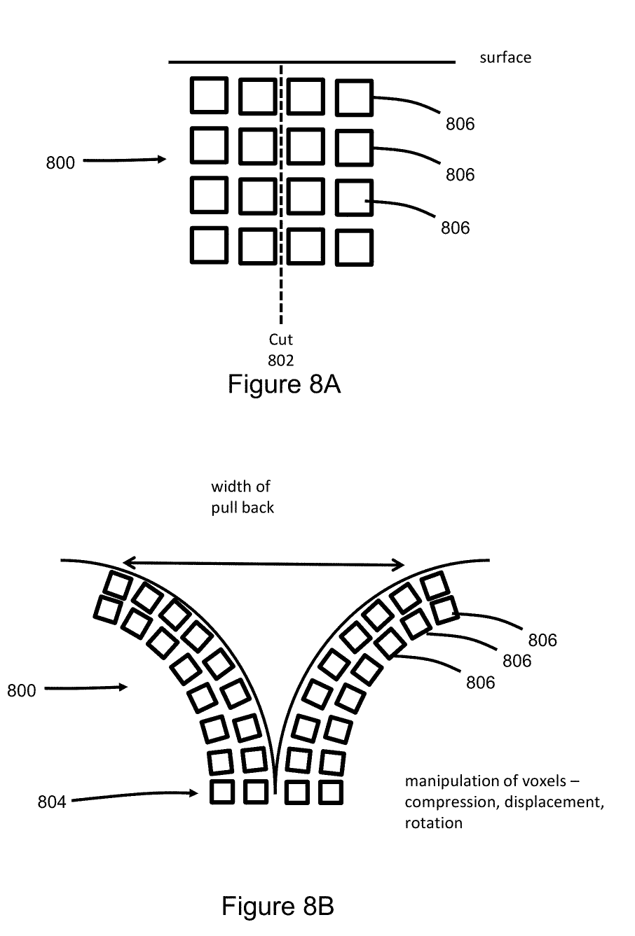

Referring to FIGS. 8A and 8B, during an operation, the surgeon cuts into the patient and displaces tissue 800 along the cut 802 to reach tissue 804 which is the objective of the operation. The displaced tissue 800 is not destroyed, but instead pulled to each side of the cut 802. As a result, the original 3D pre-operative medical imaging data set is no longer valid in the region of the cut. A representation of this displaced tissue is termed deformable tissue and it applies to the 3D data. The degree of deformation is based on the depth and length of the cut, the type of tissue adjacent to the cut, and the width the surgeon chooses to pull back the tissue. The deformation models (e.g., voxel displacement, re-sizing, re-orienting, adding new voxels, subtracting voxels, etc.) will be inputted into a real-time 3D medical imaging dataset for viewing on the HDU, recording and analysis. Adjustment of the voxels 806 of this deformable tissue are illustrated in these figures.

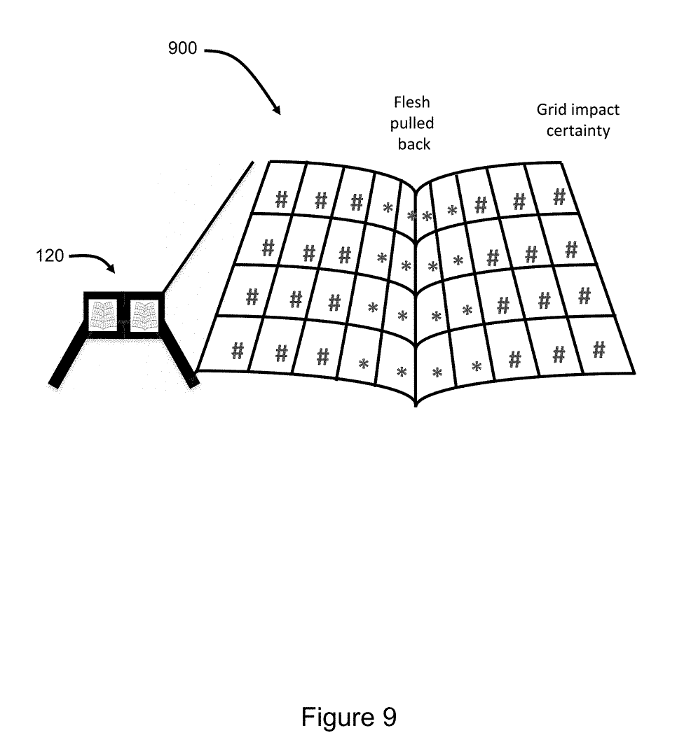

FIG. 9 illustrates exposed deformable tissue 900 from a top view as viewed through the HDU 120 at the surgeon's command. A metric grid may be superimposed on the image or patient to facilitate the surgeons understanding the cut depth at any time during the operation. Color coding may be used to indicate proximity to tissue which is the objective of the operation. For example: the tissue in the early stages at several centimeters from the objective tissue could be tinted light green. This would signify to the surgeon that the cutting could continue at the desired pace for this distance. The color could progressively change from light green to yellow as the cutting nears the objective tissue. Finally, changes to blue in close proximity to the objective tissue. Red areas would be designated as areas to avoid.

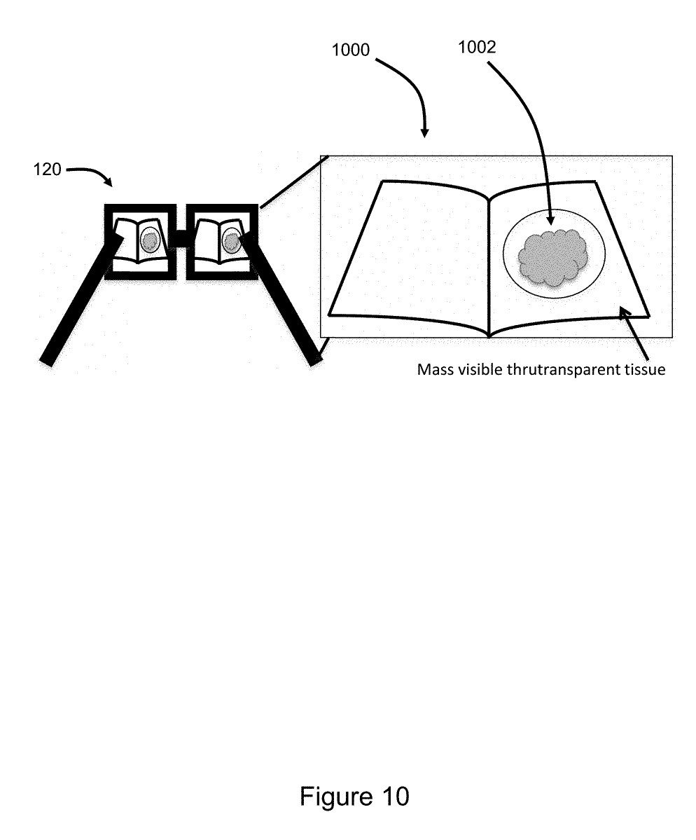

FIG. 10 illustrates exposed deformable tissue 1000 from a top view as viewed through the HDU 120. In the illustrated mode a variable degree of transparency is selected so that the surgeon can peer through the deformable tissue and see other portions of the anatomy (e.g., tumor 1002 in the deeper tissues) in the general region of the cut. The transparency may be selected at the surgeon's command. As an example, if the cut were passing through fatty tissue and this fatty tissue pulled back (i.e., deformed), then this fatty tissue could be highly transparent and the surgeon could see the near the cut surface. This view would also be useful to show the surgeon where areas of concern delineated during the pre-operation planning. False color could be added to these areas of concern (e.g., red color for arteries in proximity to the cutting surface.)

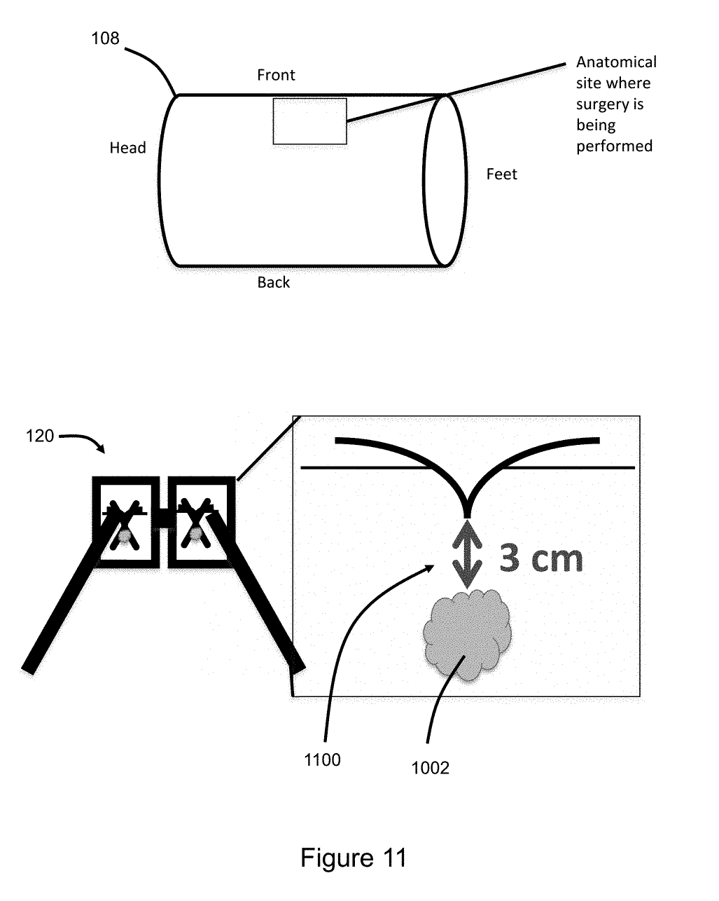

FIG. 11 illustrates a side view of the patient 108 as viewed through the HDU 120, wherein the depth 1100 of the cut is shown as a line and the tissue 1002 which is the objective of the operation is highlighted. Other portions of the body which could occlude viewing the line and the objective tissue are transparent. At this juncture, the surgeon could prompt calculation of the distance between the cut line and the objective tissue. At the surgeon's command this line, objective tissue and metric could be displayed on the surgeon's HDU 120. In a similar manner, a top view could be generated and metrics calculated to area of concern. This too would be available for display on the HDU.

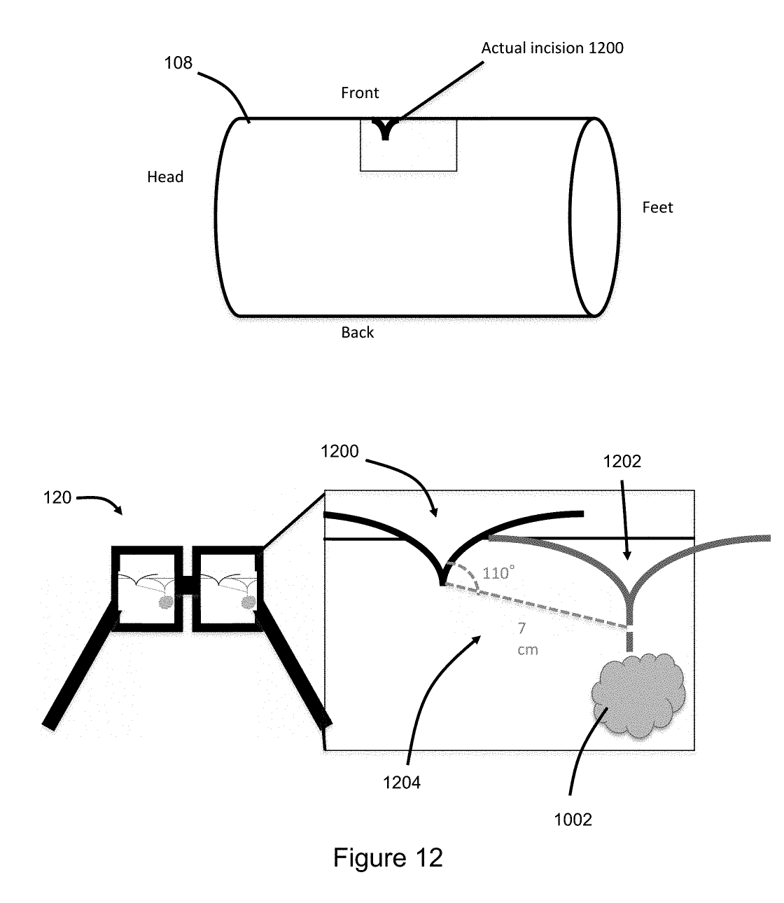

FIG. 12 illustrates a condition wherein the actual incision 1200 has deviated from the planned incision 1202 as viewed through the HDU 120. This would be computed continually. Metrics that describe acceptable deviation limits may be specified, and if the actual deviation exceeds the specific limits then the surgeon would be alerted, e.g. via the HDU 120. At this juncture, the surgeon could choose to display on the HDU the two cutting surfaces (actual cutting surface and planned cutting surface). As a further assist to the surgeon, a corrective cut 1204 to reach the desired point on the objective tissue may be calculated and displayed on the HDU. Several options for display of the corrective cutting angle include a roll angle for the surgical device. This could be continuously calculated and displayed as the SD inertial motion sensor system noted changes in the roll angle and displayed further changes, as necessary. The HDU may also be used to request and display advice from an Artificial Intelligence (AI) program running on computer 110 (FIG. 1) or elsewhere. For example, the AI program might be integrated with the control elements program 112 (FIG. 1), or available via the Internet. The AI program could be called by the surgeon, for example, if an artery were severed. The surgeon could ask the AI program for corrective actions, and corrective actions suggested by the AI could be presented by the HDU, e.g. possibly including visual and auditory information.

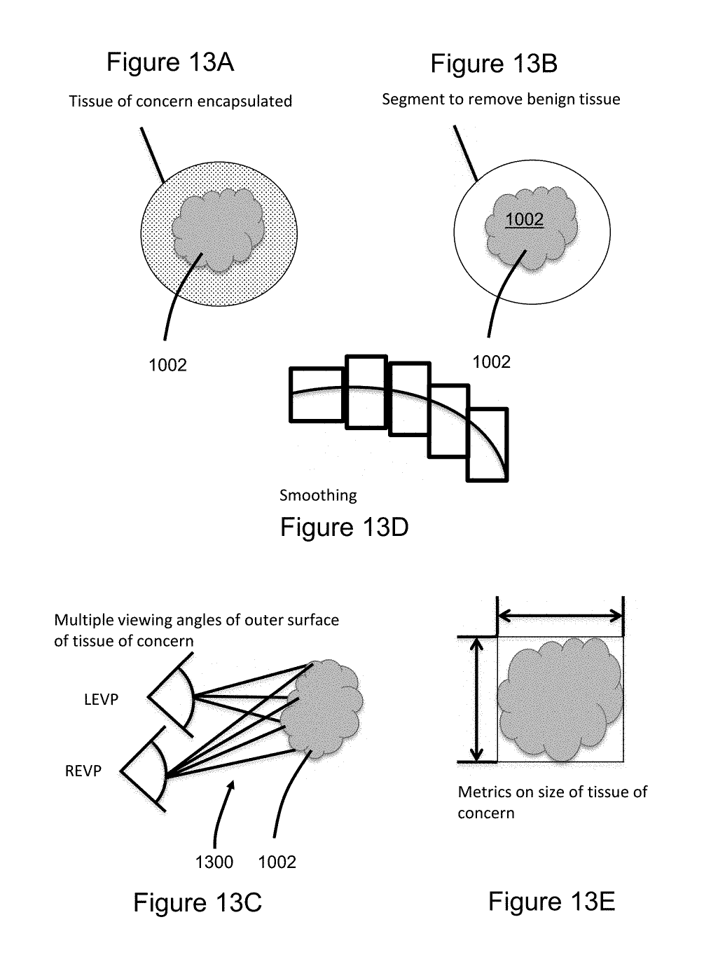

FIG. 13A illustrates encapsulation of the tissue of concern 1002 for the operation with a margin of benign tissue surrounding the tissue of concern within this encapsulation. The segmentation process is then applied as shown in FIG. 13 B, and then tissue which is extraneous to the to the operation is then subtracted from the encapsulated volume as viewed through the HDU. Thus, only the tissue of concern remains in the encapsulated volume. At this juncture, a process is undertaken to ascertain which voxels are on the outer surface of the volume which contains the tissue of concern for the operation. This involves both the left eye view (LEVP) point and the right eye view point (REVP) as shown in FIG. 13C. For each of these viewpoints, rays 1300 are drawn which intersect with the volume and, for each ray, the minimum distance is recorded. This yields a surface which is generally convex and oriented toward the surgeon. If this process is conducted from multiple viewpoints, then a volume which represents the outer surface of the tissue of concern is established. At this juncture a smoothing algorithm may be applied wherein anomalies are largely eliminated through techniques such as Fourier transforms as shown in FIG. 13D. The resulting volume can then be displayed to the surgeon on the HMDs. Metrics would be available to show the dimensions of this volume as illustrated in FIG. 13E. The shape of the volume would be readily apparent, and this could guide the conduct of the surgical procedure.

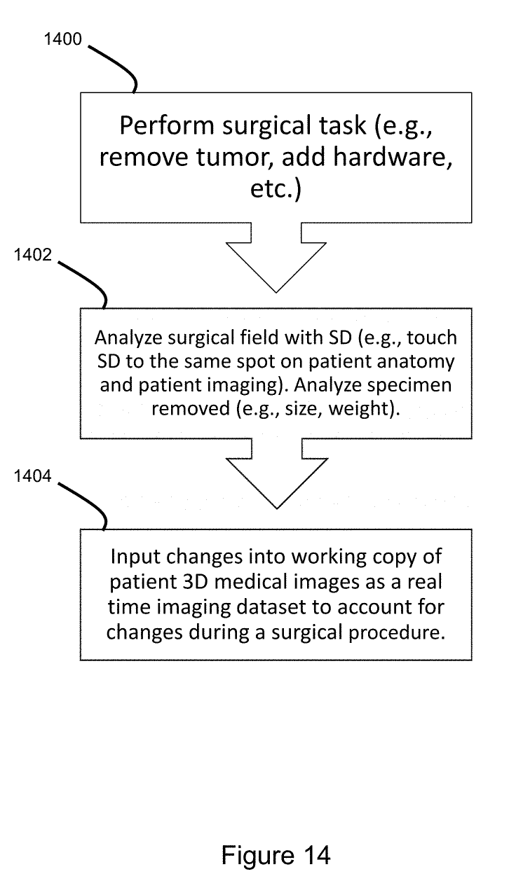

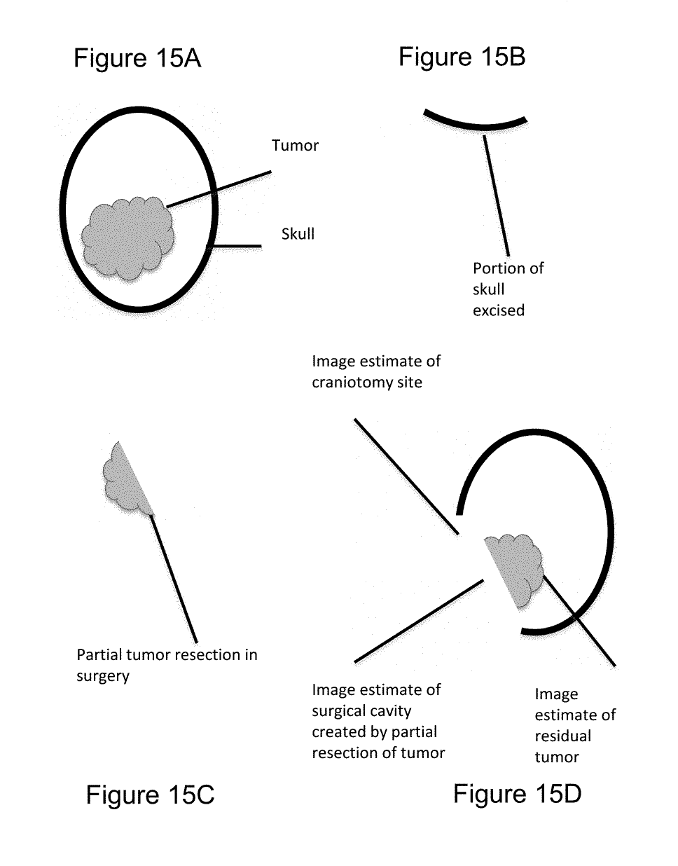

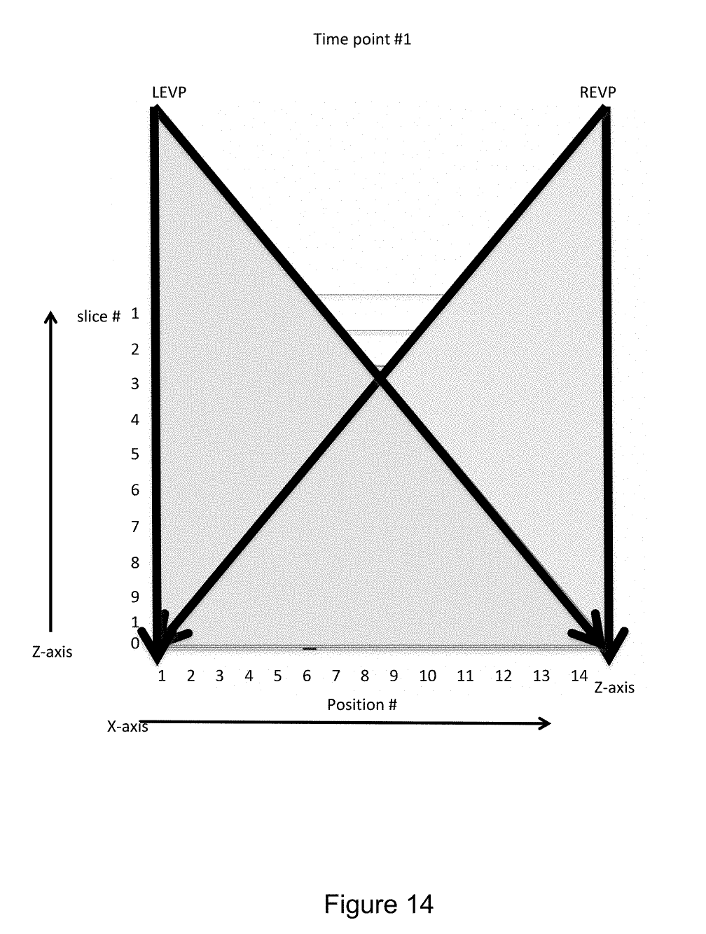

FIG. 14 illustrates a process for generating a real-time imaging dataset to better approximate the current surgical anatomy with reference to FIGS. 15A through 15D. Initially, a real-time imaging dataset will be generated as part of the pre-operative imaging examination as shown in FIG. 15A. The surgeon performs a surgical task as shown in block 1400, such as removing a portion of the skull and a portion of a tumor. Next, the surgeon and medical team will analyze the surgical bed with the SD and resected elements as shown in block 1402 to generate size, shapes, weights, tissue components of the removed elements as shown in FIGS. 15B and 15C. The shape of the surgical cavity will be determined. Next, the matched volumes are removed from the medical imaging dataset as shown in FIG. 15D. The resulting image will be a modified real time of the actual patient anatomy during the surgery as shown in block 1404. In other surgical procedures, hardware is added. In these such situations, a digital 3D representation of the surgical hardware is superimposed into the medical image. The voxels will be stretched accordingly.

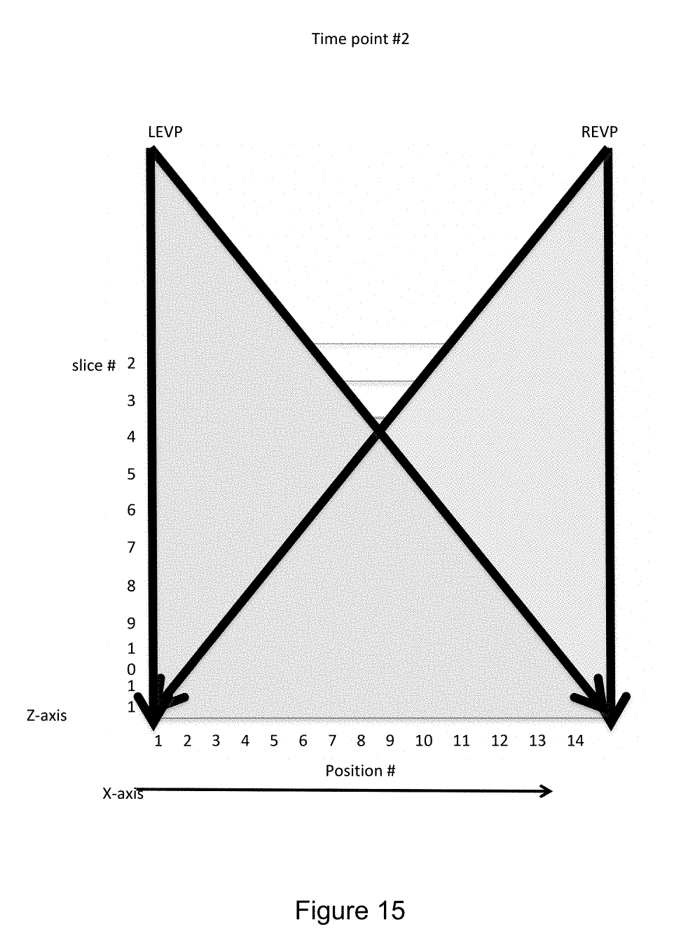

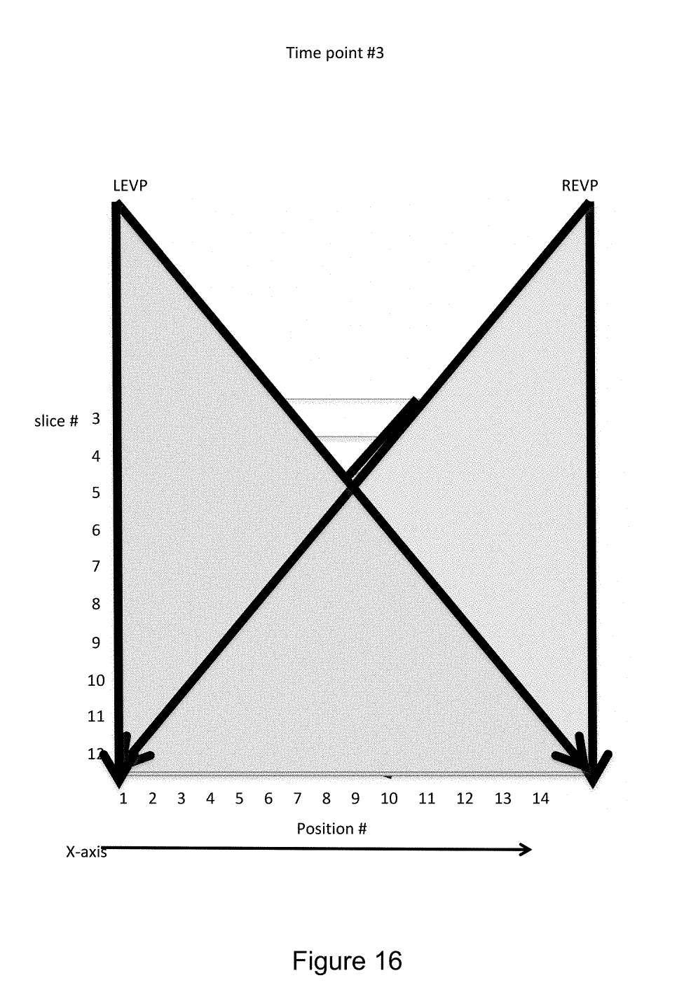

FIGS. 16 through 18 illustrate stacking of slices to generate a mobile volume. In cases where the tissue anatomy is complex, the medical professional can have the ability to isolate the volume of patient tissues displayed down to a small number of slices (e.g., coronal slices) to form a stack. The initial head position displays slices 1-10. As the head position is moved toward the surgical field, the displayed images would include slices 2-11, then 3-12 and so on. This implementation of a mobile volume displayed allows the surgeon to view a complex structure, piece by piece. Although a progression of one slice per subsequent view was illustrated, the progression could be multiple slices with each progressive step. The degree of stepping would be controlled by the medical professional.

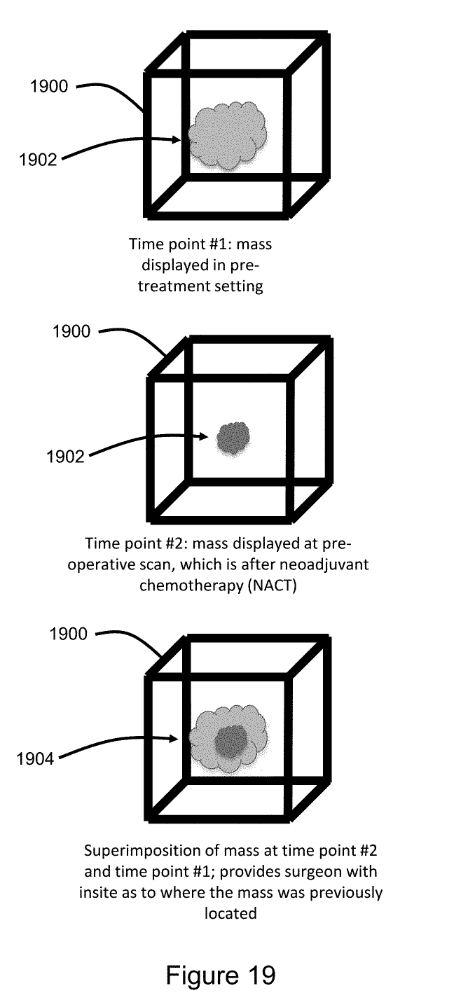

FIG. 19 illustrates a 4-dimensional (4D) cursor 1900 with dimensions including length, width, height and time. Since a cancer mass 1902 can change in shape and size over time in its natural course (i.e., growth) or in response to neoadjuvant chemotherapy (NACT) (i.e., ideally shrink), a surgeon may be interested in the size and extent of the tumor at multiple time points. Therefore, implementations will include displaying the mass in 3D at the time of diagnosis, after NACT, or superimposition 1904 of multiple time points in a single 3D image.

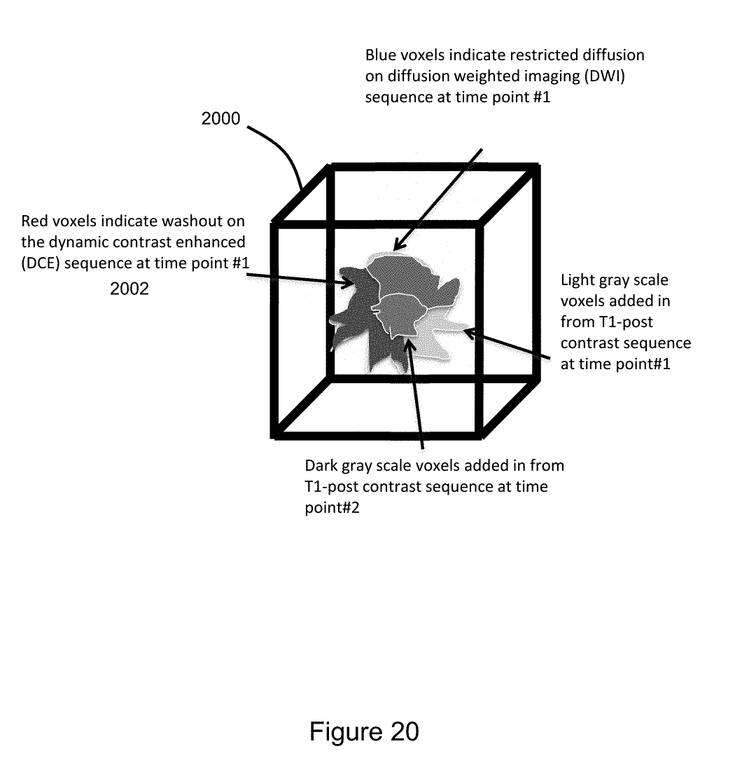

FIG. 20 illustrates a 5+ multidimensional cursor 2000. In addition to the standard volume dimensions (length, width, height), additional user-selected dimensions will be provided. Since Mill imaging provides multiple sequences (e.g., T1-weighted, T2-weighted, diffusion weighted imaging (DWI), dynamic contrast enhanced (DCE), properties of each of these images can be selected to be displayed in the surgeon's augmented reality headset. Specifically, the areas of enhancement with washout kinetics 2002, which are concerning for tumor are color coded red. The surgeon may deem this to be the most dangerous portion of the tumor and may elect to take the widest margin at this location.

Several features, aspects, embodiments and implementations have been described. Nevertheless, it will be understood that a wide variety of modifications and combinations may be made without departing from the scope of the inventive concepts described herein. Accordingly, those modifications and combinations are within the scope of the following claims.

What is claimed is:

1. An apparatus comprising:
a plurality of spatial locators adapted to be used in an operating room;
a medical image registration device configured to use information from the spatial locators to register at least one medical image with respect to a human body in the operating room that will undergo a surgical procedure; and
a display that presents the registered medical image.

2. The apparatus of claim 1 wherein the spatial locators comprise transmitters and receivers, and wherein the medical image registration device calculates a distance between transmitter/receiver pairs based on elapsed time between signal transmission and signal receipt.

3. The apparatus of claim 1 wherein the spatial locators comprise a pointer with an inertial sensor and a plurality of reference markers adapted to be positioned proximate to the body.

4. The apparatus of claim 1 further comprising at least one surgical device equipped with at least one registration feature, and wherein the medical image registration device uses information from the sensors to register the at least one medical image with respect to the surgical device.

5. The apparatus of claim 2 wherein the registration feature comprises a receiver that is sensed by the plurality of sensors.

6. The apparatus of claim 2 wherein the registration feature comprises an inertial motion sensor.

7. The apparatus of claim 1 wherein the display comprises an augmented reality headset.

8. The apparatus of claim 7 wherein the augmented reality headset comprises an inertial motion sensor.

9. The apparatus of claim 7 wherein the registered medical image is overlaid on a view of the body with the augmented reality headset.

10. The apparatus of claim 9 wherein the registration markers are presented in the registered medical image.

11. The apparatus of claim 2 wherein the surgical device comprises a cutting element, and wherein the medical image registration device records location and orientation of the cutting element with respect to the registered medical image.

12. The apparatus of claim 11 wherein the medical image registration device calculates a projected future path and orientation of the cutting element with respect to the registered medical image.

13. The apparatus of claim 10 wherein a planned surgical incision is generated and stored in a pre-operative planning record, and wherein the planned surgical incision is presented in the displayed registered medical image.

14. The apparatus of claim 13 wherein a warning is generated in response to deviation of the projected future path and orientation of the cutting element from the planned surgical incision.

15. The apparatus of claim 14 wherein a corrective cut is displayed by the augmented reality headset.

16. The apparatus of claim 14 wherein an artificial intelligence program presents suggested corrective actions via the augmented reality headset.

17. The apparatus of claim 13 wherein a recorded surgical incision is presented in the displayed registered medical image.

18. The apparatus of claim 1 wherein the at least one medical image comprises a three-dimensional patient dataset generated from a plurality of two-dimensional radiological images.

19. A method comprising:
receiving data from a plurality of spatial locators adapted to be used in an operating room;
using the data from the spatial locators to register at least one medical image with respect to a human body in the operating room that will undergo a surgical procedure; and
presenting the registered medical image on a display.

20. The method of claim 19 wherein the spatial locators comprise transmitters and receivers, and comprising calculating a distance between transmitter/receiver pairs based on elapsed time between signal transmission and signal receipt.

21. The method of claim 19 wherein the spatial locators comprise a pointer with an inertial sensor and a plurality of reference markers adapted to be positioned proximate to the body, and comprising calculating a distance between a registration point and the reference markers.

22. The method of claim 19 further comprising at least one surgical device equipped with at least one registration feature, and comprising using information from the sensors to register the at least one medical image with respect to the surgical device.

23. The method of claim 20 comprising the plurality of sensors sensing the registration feature.

24. The method of claim 20 comprising the registration feature sensing motion.

25. The method of claim 19 comprising presenting the registered medical image on an augmented reality headset.

26. The method of claim 25 wherein the augmented reality headset comprises an inertial motion sensor, and comprising registering the spatial location of the augmented reality headset with respect to the at least one medical image based on information from the inertial motion sensor.

27. The method of claim 26 comprising overlaying the registered medical image on a view of the body with the augmented reality headset.

28. The method of claim 27 comprising presenting the registration markers in the registered medical image.

29. The method of claim 20 wherein the surgical device comprises a cutting element, and comprising recording location and orientation of the cutting element with respect to the registered medical image.

30. The method of claim 29 comprising calculating a projected future path and orientation of the cutting element with respect to the registered medical image.

31. The method of claim 30 comprising generating and storing a planned surgical incision in a pre-operative planning record, and presenting the planned surgical incision in the displayed registered medical image.

32. The method of claim 31 comprising generating a warning in response to deviation of the projected future path and orientation of the cutting element from the planned surgical incision.

33. The method of claim 31 comprising presenting a recorded surgical incision in the displayed registered medical image.

34. The method of claim 32 comprising displaying a corrective cut with the augmented reality headset.

35. The method of claim 32 comprising an artificial intelligence program presenting suggested corrective actions via the augmented reality headset.

36. The method of claim 19 comprising generating the at least one medical image as a three-dimensional patient dataset from a plurality of two-dimensional radiological images.

37. The method of claim 29 comprising generating a real time updated set of radiological images based on surgical changed to the patient's anatomy.
